(19) United States
(12) Patent Application Publication
(10) Pub. No.: US 2025/0017647 A1
(43) Pub. Date: Jan. 16, 2025

(54) RADIO FREQUENCY CATHETER

(71) Applicant: Acotec Scientific Co., Ltd., Beijing (CN)

(72) Inventors: Jian GUO, Beijing (CN); Dawei CAO, Beijing (CN); Cong ZHANG, Beijing (CN)

(73) Assignee: Acotec Scientific Co., Ltd., Beijing (CN)

(21) Appl. No.: 18/664,566

(22) Filed: May 15, 2024

(30) Foreign Application Priority Data
Jul. 14, 2023 (CN) 202310862927.5

(51) Int. Cl. A61B 18/14; A61B 18/00

(52) U.S. Cl. CPC A61B 18/1492; A61B 2018/00101; A61B 2018/00404; A61B 2018/00577; A61B 2018/00821; A61B 2018/1407

(57) ABSTRACT

The present disclosure provides an RF catheter, including: a catheter body; and at least one heating unit and at least one electrode fixedly coupled to the catheter body. The at least one heating unit is configured to perform ablation on a target tissue in a superficial vein, the at least one electrode is configured to perform ablation on a target issue in a perforating vein, each of the heating unit and the electrode is in a corresponding target operating mode in accordance with an operating mode command for the RF catheter, and the target operating modes include a first operating mode where the electrode operates, a second operating mode where at least two heating units operate, and a third operating mode where merely one heating unit operates.

### TECHNICAL FIELD

The present disclosure relates to the field of Radio Frequency (RF) treatment technology, in particular to an RF catheter.

### BACKGROUND

For the treatment of varicose veins of a lower limb, RF closure is a principal method. However, there mainly exist the following problems in an RF catheter for the RF closure.

1. It is impossible for a single RF catheter to complete the RF closure for a superficial vein and a perforating vein of the lower limb simultaneously. During the surgery, different instruments need to be used, leading to a risk during the change of the instruments. In addition, an operation time is prolonged, and an economic burden of a patient increases.

2. The RF catheter is provided with a single heating coil. When the heating coil is long, the ablation efficiency is high, but it is impossible to perform the treatment on a short vein, a small saphenous vein, an accessory saphenous vein, and a branch/perforating branch. When the heating coil is short, it is able to perform the treatment on the short vein, but the ablation efficiency is low, and the operation time is prolonged significantly.

### SUMMARY

An object of the present disclosure is to provide an RF catheter, so as to complete the RF closure on the superficial vein and the perforating vein of the lower limb through a single RF catheter simultaneously.

In order to solve the above-mentioned problem, the present disclosure provides the following technical solutions.

The present disclosure provides in some embodiments an RF catheter, including: a catheter body; and at least one heating unit and at least one electrode fixedly coupled to the catheter body. The at least one heating unit is configured to perform ablation on a target tissue in a superficial vein, the at least one electrode is configured to perform ablation on a target issue in a perforating vein, each of the heating unit and the electrode is in a corresponding target operating mode in accordance with an operating mode command for the RF catheter, and the target operating modes include a first operating mode where the electrode operates, a second operating mode where at least two heating units operate, and a third operating mode where merely one heating unit operates.

In a possible embodiment of the present disclosure, the at least one heating unit includes a first heating unit and a second heating unit arranged side by side, a length of the first heating unit is smaller than or equal to a length of the second heating unit, the first heating unit is arranged adjacent to the electrode, and the second heating unit is spaced apart from the first heating unit by a first predetermined distance.

In a possible embodiment of the present disclosure, each of the first heating unit and the second heating unit is a coil; a first thermocouple is arranged in and insulated from the first heating unit, and configured to measure a temperature of the first heating unit; and a second thermocouple is arranged in and insulated from the second heating unit, and configured to measure a temperature of the second heating unit.

In a possible embodiment of the present disclosure, each of the first heating unit and the second heating unit is a metallic tube, a groove is formed in a surface of the metallic tube, two pins extend from the metallic tube, and a thermocouple is arranged in the groove and configured to measure a temperature of the first heating unit or the second heating unit.

In a possible embodiment of the present disclosure, when each of the first heating unit and the second heating unit is a coil, the RF catheter further includes a winding tube fixedly coupled to the catheter body, and the coils of the first heating unit and the second heating unit are wound around the winding tube.

In a possible embodiment of the present disclosure, the first thermocouple and the second thermocouple are adhered onto a surface of the winding tube, the first thermocouple is arranged between adjacent coil threads of the first heating unit, and the second thermocouple is arranged between adjacent coil threads of the second heating unit.

In a possible embodiment of the present disclosure, the at least one electrode includes a first electrode and a second electrode arranged side by side, the first electrode is arranged adjacent to the first heating unit and spaced apart from the first heating unit by a second predetermined distance, the first electrode is spaced apart from the second electrode by a third predetermined distance, and a third thermocouple is fixed onto an inner wall of the first electrode or the second electrode and configured to measure a temperature of the first electrode or the second electrode.

In a possible embodiment of the present disclosure, an RF energy transmission line and a thermocouple compensation line are arranged inside the catheter body, the RF energy transmission line is coupled to the first heating unit, the second heating unit, the first electrode and the second electrode, and the thermocouple compensation line is coupled to the first thermocouple, the second thermocouple and the third thermocouple.

In a possible embodiment of the present disclosure, the RF catheter further includes at least one of: a thermally-shrinkable tube which is a hollow cylinder enclosing and being in contact with the first heating unit and the second heating unit; a circular plug arranged at an end of the second electrode; a handle coupled to the catheter body, an integrated circuit board being arranged inside the handle; an integrated cable extending from the handle; or a cable taper coupled to an end of the integrated cable and an RF energy generator.

In a possible embodiment of the present disclosure, the catheter body is a hollow, cylindrical catheter provided with at least one marking tape, and a locating ring is sleeved onto and in contact with the catheter body.

The present disclosure at least has the following beneficial effect.

According to the embodiments of the present disclosure, the RF catheter includes: the catheter body; and the at least one heating unit and the at least one electrode fixedly coupled to the catheter body. The at least one heating unit is configured to perform ablation on the target tissue in the superficial vein, the at least one electrode is configured to perform ablation on the target issue in the perforating vein, each of the heating unit and the electrode is in a corresponding target operating mode in accordance with the operating mode command for the RF catheter, and the target operating modes include the first operating mode where the electrode operates, the second operating mode where at least two heating units operate, and the third operating mode where merely one heating unit operates. As a result, it is able to complete the RF closure on the superficial vein and the perforating vein of the lower limb through a single RF catheter simultaneously.

### BRIEF DESCRIPTION OF THE DRAWINGS

FIG. 1 is a schematic view showing an RF catheter according to one embodiment of the present disclosure;

FIG. 2 is a schematic view showing a tip of the RF catheter according to one embodiment of the present disclosure;

FIG. 3 is a schematic view showing a heating metallic tube for pins of the RF catheter at different sides according to one embodiment of the present disclosure;

FIG. 4 is a schematic view showing a heating metallic tube for pins of the RF catheter at a same side according to one embodiment of the present disclosure;

FIG. 5 is a schematic view showing a heating coil and a thermocouple of the RF catheter according to one embodiment of the present disclosure;

FIG. 6 is a schematic view showing an electrode and the thermocouple of the RF catheter according to one embodiment of the present disclosure;

FIG. 7 is a schematic view showing the assembling of the electrode and the thermocouple of the RF catheter through a groove according to one embodiment of the present disclosure;

FIG. 8 is a schematic view showing the tip of the RF catheter with a single electrode according to one embodiment of the present disclosure;

FIG. 9 is a schematic view showing the tip of the RF catheter with a guide-wire cavity according to one embodiment of the present disclosure;

FIG. 10 is another schematic view showing the tip of the RF catheter with the guide-wire cavity according to one embodiment of the present disclosure; and

FIG. 11 is yet another schematic view showing the tip of the RF catheter with the guide-wire cavity according to one embodiment of the present disclosure.

### REFERENCE SIGN LIST

1 catheter body
11 locating ring
12 guide-wire cavity
13 Luer taper
21 first heating unit
22 second heating unit
23 first thermocouple
31 first electrode
32 second electrode
33 third thermocouple
34 annular electrode
41 RF energy transmission line
42 thermocouple compensation line
5 winding tube
6 thermally-shrinkable tube
7 circular plug
8 handle
9 integrated cable
10 cable taper

### DETAILED DESCRIPTION

The present disclosure will be described hereinafter in conjunction with the drawings and embodiments. The following embodiments are for illustrative purposes only, but shall not be used to limit the scope of the present disclosure. Actually, the embodiments are provided so as to facilitate the understanding of the scope of the present disclosure.

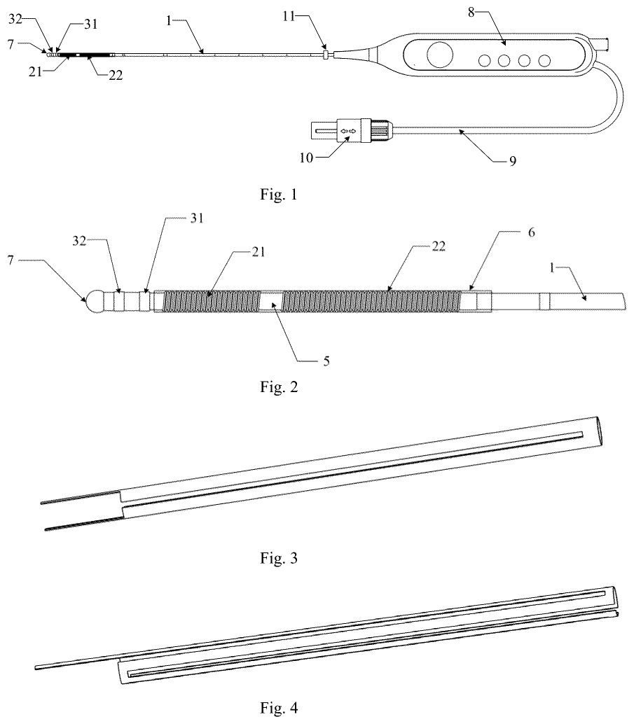

As shown in FIG. 1, the present disclosure provides in some embodiments an RF catheter, which includes: a catheter body 1; and at least one heating unit and at least one electrode fixedly coupled to the catheter body 1. The at least one heating unit is configured to perform ablation on a target tissue in a superficial vein, the at least one electrode is configured to perform ablation on a target issue in a perforating vein, each of the heating unit and the electrode is in a corresponding target operating mode in accordance with an operating mode command for the RF catheter, and the target operating modes include a first operating mode where the electrode operates, a second operating mode where at least two heating units operate, and a third operating mode where merely one heating unit operates.

In the embodiments of the present disclosure, the heating unit and the electrode are in the corresponding target operating mode in accordance with the operating mode command for the RF catheter, so as to complete RF closure on a superficial vein and a perforating vein of a lower limb. The catheter is provided with one or two electrodes at a distal end for the RF closure on the perforating vein. A plurality of heating units is integrated on the catheter and distributed axially for the RF closure on the superficial vein. Each heating unit is controlled independently and outputs energy independently, and in use, the operating mode of the RF catheter is selected dynamically according to the therapeutic demand. Through the plurality of heating units, it is able to perform the ablation on a long vein segment and a short vein segment through a single catheter, without any attenuated ablation efficiency.

As shown in FIG. 2, in a possible embodiment of the present disclosure, the at least one heating unit includes a first heating unit 21 and a second heating unit 22 arranged side by side, a length of the first heating unit 21 is smaller than or equal to a length of the second heating unit 22, the first heating unit 21 is arranged adjacent to the electrode, and the second heating unit 22 is spaced apart from the first heating unit 21 by a first predetermined distance.

In the embodiments of the present disclosure, an energy application member of the RF catheter is provide with two heating units. During the treatment, the first heating unit 21 and the second heating unit 22 generate heat and supply the heat to a position where a lesion occurs, i.e., energy is supplied to a position where a lesion of a blood vessel through RF heating. Merely the first heating unit 21, or both the first heating unit 21 and the second heating unit 22, are selected to operate in accordance with the position where the lesion occurs. Each of the heating units is controlled independently and output energy independently. In use, the heating units are selected dynamically. When a current passes through the heating unit, a high temperature occurs due to high resistivity, and the heat is transferred to an inner wall of the blood vessel for ablation. Through the plurality of heating units, it is able to perform the ablation on a long vein segment and a short vein segment through a single catheter, without any attenuated ablation efficiency.

In a possible embodiment of the present disclosure, each of the first heating unit 21 and the second heating unit 22 is a coil. A first thermocouple 23 is arranged in and insulated from the first heating unit 21, and configured to measure a temperature of the first heating unit 21. A second thermocouple is arranged in and insulated from the second heating unit 22, and configured to measure a temperature of the second heating unit 22.

In the embodiments of the present disclosure, each of the first heating unit 21 and the second heating unit 22 is obtained through winding a high-resistivity metallic wire with an insulation layer. In order to reduce an inductance of the coil itself, a single metallic wire is folded and then wound in a bifilar manner. The metallic wire is made of a copper-nickel alloy, a nickel-chromium alloy, a nickel-chromium-iron alloy or an iron-chromium-aluminium alloy. The insulation layer is made of high density polyethylene (HDPE), polytetrafluoroethylene (PTFE) or polyimide (PI). The metallic wire has a diameter of 0.05 mm to 0.2 mm. The coils are spaced apart from each other by a distance of 0.5 mm to 10 mm, so as to facilitate the identification of different coils under an ultrasonic wave.

Each of the first heating unit 21 and the second heating unit 22 is provided with a thermocouple, so as to measure a real-time temperature of the coil, and transmit the real-time temperature to an RF generator, thereby to control the temperature of the heating unit. The RF generator is coupled to the catheter via a cable, and configured to provide energy to a tip of the catheter during the operation of the RF catheter.

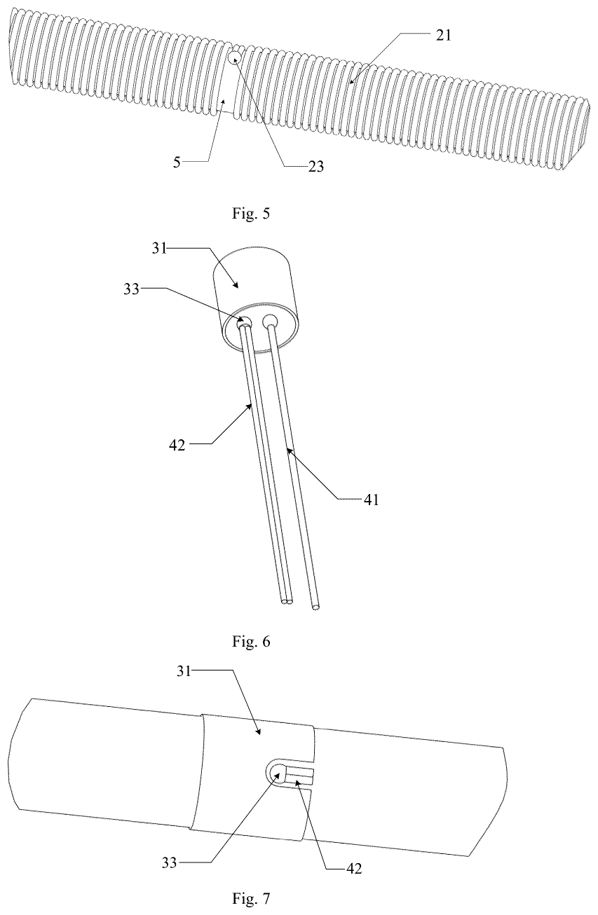

As shown in FIGS. 3 and 4, in a possible embodiment of the present disclosure, each of the first heating unit 21 and the second heating unit 22 is a metallic tube, a groove is formed in a surface of the metallic tube, two pins extend from the metallic tube, and a thermocouple is arranged in the groove and configured to measure a temperature of the first heating unit 21 or the second heating unit 22.

In the embodiments of the present disclosure, apart from the above, each of the first heating unit 21 and the second heating unit 22 may also be provided in the other form, e.g., a high-impedance metallic tube with a groove in a special form and two pins. The two pins are coupled to an enameled wire, so as to transmit an RF electric signal. The thermocouple is placed in the groove. The groove is provided in a symmetrical form as shown in FIG. 3, and at this time, the two pins are located at different sides on one end of the metallic tube. In addition, the groove extends back and forth from one pin to another pin as shown in FIG. 4, and at this time, the two pins are located at a same side on one end of the metallic tube. Of course, in the embodiments of the present disclosure, an outer surface of the high-impedance metallic tube is provided with patterns, so as to increase the flexibility and compliance of the metallic tube, thereby to facilitate the intervention of the RF catheter to the position where the lesion of the blood vessel occurs.

As shown in FIG. 5, in a possible embodiment of the present disclosure, when each of the first heating unit 21 and the second heating unit 22 is a coil, the RF catheter further includes a winding tube 5 fixedly coupled to the catheter body 1, and the coils of the first heating unit 21 and the second heating unit 22 are wound around the winding tube 5.

In the embodiments of the present disclosure, the RF catheter further includes the winding tube 5, and the winding tube 5 is fixedly coupled to the catheter body 1 and configured to fix the coils.

As shown in FIG. 5, in a possible embodiment of the present disclosure, the first thermocouple 23 and the second thermocouple are adhered onto a surface of the winding tube 5, the first thermocouple 23 is arranged between adjacent coil threads of the first heating unit 21, and the second thermocouple is arranged between adjacent coil threads of the second heating unit 22.

In the embodiments of the present disclosure, the coil is wound around the winding tube 5, and the first thermocouple 23 or the second thermocouple is adhered onto the surface of the winding tube 5 at a position where a larger thread pitch is provided. The first thermocouple 23 or the second thermocouple is not in contact with the coil, i.e., it is insulated from the coil, with an insulation impedance not smaller than 10 MΩ at a voltage of 500V. In order to increase a heat conduction rate, an insulative, thermally-conductive adhesive is applied between the first thermocouple 23 or the second thermocouple and the coil.

In a possible embodiment of the present disclosure, the at least one electrode includes a first electrode 31 and a second electrode 32 arranged side by side. The first electrode 31 is arranged adjacent to the first heating unit 21 and spaced apart from the first heating unit 21 by a second predetermined distance. The first electrode 31 is spaced apart from the second electrode 32 by a third predetermined distance, and a third thermocouple 33 is fixed onto an inner wall of the first electrode 31 or the second electrode 32 and configured to measure a temperature of the first electrode 31 or the second electrode 32.

As shown in FIG. 2, in the embodiments of the present disclosure, the first electrode 31 and the second electrode 32 are arranged side by side at the tip of the catheter. The second electrode 32, the first electrode 31, the first heating unit 21 and the second heating unit 22 are arranged side by side at intervals. Each of the first electrode 31 and the second electrode 32 is of a ring shape, and made of a platinum-iridium alloy or stainless steel. The first electrode 31 is insulated from the second electrode 32. During the operation, the first electrode 31 and the second electrode 32 are of different polarities, and an RF current flows from a positive electrode through a human body to a negative electrode, so as to generate the heat and apply the heat onto an inner wall of the blood vessel. Each of the electrodes has an outer diameter of 1 mm to 2.5 mm and a length of 0.5 mm to 2 mm, and the two electrodes are spaced apart from each other by 0.2 mm to 2 mm.

As shown in FIG. 6, an RF signal cable is coupled to the inner wall of each of the first electrode 31 and the second electrode 32, and a thermocouple is arranged on the inner wall of one of the first electrode 31 and the second electrode 32 for measuring the temperature. The third thermocouple 33 is fixed onto the inner wall of the first electrode 31 or the second electrode 32. Before the fixation, a thermally-shrinkable tube or a PI tube is sleeved onto the thermocouple at a temperature-measuring end, so that the thermocouple is insulated from the electrode. The fixation is achieved through a UV-curable adhesive or a thermally-conductive adhesive. An RF energy transmission line 41 is fixed, through soldering, onto the inner wall of the electrode, so as to transmit an RF electric signal. The soldering includes laser soldering, ultrasonic soldering or tin soldering.

As shown in FIG. 7, in another possible embodiment of the present disclosure, the third thermocouple 33 is further fixed into a groove of the electrode. During the manufacture, the first electrode 31 or the second electrode 32 is a metallic ring made of platinum-iridium alloy or stainless steel, a groove is formed in the metallic ring through a femtosecond laser, and then the third thermocouple 33 is placed into the groove and fixed to the electrode through a thermally-conductive adhesive.

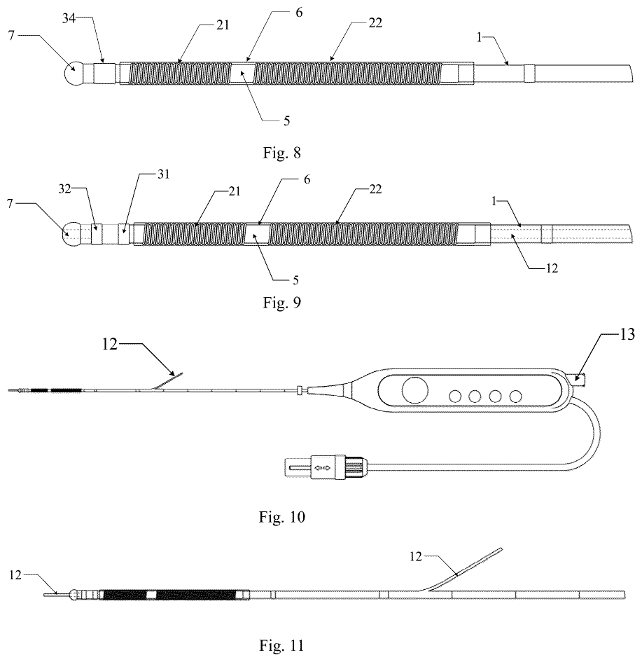

FIG. 8 shows an RF catheter with a single electrode, i.e., the RF catheter is provided with merely one annular electrode 34 for energy output. During the treatment, a conductive back plate is adhered onto the human body, so as to form a loop with the annular electrode at the tip of the catheter. When the RF current flows through the human body, heat is generated surrounding the annular electrode, so as to perform the ablation on the inner wall of the blood vessel.

In a possible embodiment of the present disclosure, an RF energy transmission line 41 and a thermocouple compensation line 42 are arranged inside the catheter body 1, the RF energy transmission line 41 is coupled to the first heating unit 21, the second heating unit 22, the first electrode 31 and the second electrode 32, and the thermocouple compensation line 42 is coupled to the first thermocouple 23, the second thermocouple and the third thermocouple 33.

In the embodiments of the present disclosure, the RF energy transmission line 41 and the thermocouple compensation line 42 are arranged in the catheter body 1. The RF energy transmission line 41 is configured to transmit energy to the energy application member at the tip of the catheter, and the thermocouple compensation line 42 is configured to transmit a temperature signal.

As shown in FIG. 1, in a possible embodiment of the present disclosure, the RF catheter further includes at least one of: a thermally-shrinkable tube 6 which is a hollow cylinder enclosing and being in contact with the first heating unit 21 and the second heating unit 22; a circular plug 7 arranged at an end of the second electrode 32; a handle 8 coupled to the catheter body 1, an integrated circuit board being arranged inside the handle 8; an integrated cable 9 extending from the handle 8; or a cable taper coupled to an end of the integrated cable 9 and the RF energy generator.

In the embodiments of the present disclosure, the thermally-shrinkable tube 6 encloses the first heating unit 21 and the second heating unit 22, and it is made of fluoroplastics, e.g., fluorinated ethylene propylene (FEP) or PTFE. The thermally-shrinkable tube 6 has a thickness of 0.05 mm to 0.5 mm, and it is sealed with an UV-curable adhesive at both ends. During the operation of the RF catheter, the thermally-shrinkable tube 6 is used to prevent the RF catheter from be adhered to a human tissue, and insulate the coil from the human tissue.

The circular plug 7 is arranged at a distal end of the catheter. In an interventional operation, the circular plug 7 is in direct contact with the inner wall of the blood vessel at first, so the circular plug 7 is provided with a smooth surface, so as to prevent any damage to the inner wall of the blood vessel. In addition, through the circular plug 7, it is able to prevent a body fluid from entering the catheter, thereby to prevent the occurrence of short circuit. The circular plug 7 is made of UV-curable adhesive or thermoplastic polyurethane (TPU).

The handle 8 is provided with a switch and a plurality of indicators. The switch is configured to dynamically select an operating state of each heating unit, and start or stop the application of the RF energy. The indicator is configured to indicate a current operating state of the catheter, so as to facilitate the operation.

The integrated cable 9 includes a plurality of RF energy transmission lines and thermocouple compensation lines, and it is enclosed by an insulation layer. The integrated cable 9 is configured to transmit an RF current, a temperature signal, and a switch triggering signal or a gear position adjustment signal.

The cable taper 10 is coupled to an interface of the RF energy generator, so that the RF energy is supplied by the RF energy generator to the catheter, and the real-time temperature of the catheter is transmitted to the RF energy generator. In this way, it is able to control the magnitude of the outputted RF energy, thereby to maintain the energy application member of the catheter at a certain temperature.

In a possible embodiment of the present disclosure, the catheter body 1 is a hollow, cylindrical catheter provided with at least one marking tape, and a locating ring 11 is sleeved onto and in contact with the catheter body 1.

In the embodiments of the present disclosure, the catheter body 1 is of a tubular structure with an outer diameter of 1 mm to 2.5 mm. The catheter body 1 is provided with the marking tapes at different lengths and in different colors, so as to indicate a distance for which the catheter needs to move back after the treatment on a segment of the blood vessel. A plurality of cables and thermocouple compensation lines is arranged in the catheter body 1, so as to transmit the RF current and the temperature signal. The catheter body 1 is made of a medical-grade polymer, such as polyether-ether-ketone (PEEK), polyurethane (PU) or polyether block amide (PEBAX), and the marking tape is formed using a medical-grade ink or a medical-grade polymer film.

The locating ring 11 is of an annular structure capable of sliding along the catheter body, so as to indicate a position of the catheter during the treatment, thereby to prevent the catheter from moving back too long or too short after the treatment on a segment of the blood vessel. The locating ring 11 is made of a medical-grade polymer, e.g., PEEK, TPU or PE.

As shown in FIG. 9, in another possible embodiment of the present disclosure, the catheter body 1 is further provided with a guide-wire cavity 12. A guide wire extends along the guide-wire cavity 12 from a Luer taper 13 to the tip of the catheter. The guide wire is configured to control a position and a movement direction of the tip of the catheter in the blood vessel. The guide wire has a diameter of 0.014 inch, 0.018 inch, 0.025 inch or 0.038 inch.

As shown in FIGS. 10 and 11, in a possible embodiment of the present disclosure, the guide-wire cavity 12 further extends from a middle portion of the catheter body 1, so as to facilitate the exchange of the guide wire.

The operation of the RF catheter body will be described hereinafter.
(1). The catheter is delivered to the position where the lesion of the blood vessel is located through the guide wire and a sheath, so that the annular electrode is located in the perforating vein and the plurality of heating units is located in the superficial vein.
(2). The gear position is selected through the switch on the handle or a screen of the RF energy generator. The switch is long-pressed, so as to start the ablation on the perforating vein.
(3). The gear position of the heating units is selected through the switch on the handle or the screen of the RF energy generator. The switch is long-pressed, so as to start the ablation on the superficial vein surrounding the heating units. When the plurality of heating units is triggered simultaneously, it is able to improve the ablation efficiency of the catheter.
(4). The catheter is withdrawn by a certain distance in accordance with the marking tape, so as to complete the treatment on the other segments of superficial vein in a same way as that mentioned in (3).
(5). When a length of the to-be-treated segment of the superficial vein is smaller than a total length of the heating units, it is necessary to continuously adjust the gear position, and merely trigger the heating unit at the tip, thereby to treat the remaining segments of the short vein.
(6). The catheter is withdrawn from the body of the patient, and a puncture point is subjected to bandaging and hemostasis, so as to complete the operation.

According to the embodiments of the present disclosure, the catheter is provided with a single electrode or two electrodes at the distal end for the RF treatment on the perforating vein. The catheter includes the plurality of heating units arranged along an axial direction for the treatment on thee superficial vein. In this way, it is able to complete the RF closure on the superficial vein and the perforating vein of the lower limb simultaneously while ensuring the high ablation efficiency. Each heating unit is controlled independently and outputs the energy independently, and the operating mode of the RF catheter is selected dynamically in use. Through the plurality of heating units, it is able to perform the ablation on a long vein segment and a short vein segment through a single catheter, without any attenuated ablation efficiency. According to the catheter in the embodiments of the present disclosure, it is able to not only perform the RF ablation through bipolar or unipolar RF, but also perform the RF ablation through heat conduction. In addition, it is able to achieve the rapid exchange or over-the-wire (OTW) exchange of the catheter, thereby to facilitate the accurate intervention of the catheter to the position where the lesion of the blood vessel occurs.

The above embodiments are for illustrative purposes only, but the present disclosure is not limited thereto. Obviously, a person skilled in the art may make further modifications and improvements without departing from the spirit of the present disclosure, and these modifications and improvements shall also fall within the scope of the present disclosure.

What is claimed is:

1. A Radio Frequency (RF) catheter, comprising:
a catheter body; and
at least one heating unit and at least one electrode fixedly coupled to the catheter body,
wherein the at least one heating unit is configured to perform ablation on a target tissue in a superficial vein, the at least one electrode is configured to perform ablation on a target issue in a perforating vein, each of the heating unit and the electrode is in a corresponding target operating mode in accordance with an operating mode command for the RF catheter, and the target operating modes comprise a first operating mode where the electrode operates, a second operating mode where at least two heating units operate, and a third operating mode where merely one heating unit operates.

2. The RF catheter according to claim 1, wherein the at least one heating unit comprises a first heating unit and a second heating unit arranged side by side, a length of the first heating unit is smaller than or equal to a length of the second heating unit, the first heating unit is arranged adjacent to the electrode, and the second heating unit is spaced apart from the first heating unit by a first predetermined distance.

3. The RF catheter according to claim 2, wherein each of the first heating unit and the second heating unit is a coil;
a first thermocouple is arranged in and insulated from the first heating unit, and configured to measure a temperature of the first heating unit; and
a second thermocouple is arranged in and insulated from the second heating unit, and configured to measure a temperature of the second heating unit.

4. The RF catheter according to claim 2, wherein each of the first heating unit and the second heating unit is a metallic tube, a groove is formed in a surface of the metallic tube, two pins extend from the metallic tube, and a thermocouple is arranged in the groove and configured to measure a temperature of the first heating unit or the second heating unit.

5. The RF catheter according to claim 3, wherein when each of the first heating unit and the second heating unit is a coil, the RF catheter further comprises a winding tube fixedly coupled to the catheter body, and the coils of the first heating unit and the second heating unit are wound around the winding tube.

6. The RF catheter according to claim 5, wherein he first thermocouple and the second thermocouple are adhered onto a surface of the winding tube, the first thermocouple is arranged between adjacent coil threads of the first heating unit, and the second thermocouple is arranged between adjacent coil threads of the second heating unit.

7. The RF catheter according to claim 2, wherein the at least one electrode comprises a first electrode and a second electrode arranged side by side, the first electrode is arranged adjacent to the first heating unit and spaced apart from the first heating unit by a second predetermined distance, the first electrode is spaced apart from the second electrode by a third predetermined distance, and a third thermocouple is fixed onto an inner wall of the first electrode or the second electrode and configured to measure a temperature of the first electrode or the second electrode.

8. The RF catheter according to claim 7, wherein an RF energy transmission line and a thermocouple compensation line are arranged inside the catheter body, the RF energy transmission line is coupled to the first heating unit, the second heating unit, the first electrode and the second electrode, and the thermocouple compensation line is coupled to the first thermocouple, the second thermocouple and the third thermocouple.

9. The RF catheter according to claim 7, further comprising at least one of:
a thermally-shrinkable tube which is a hollow cylinder enclosing and being in contact with the first heating unit and the second heating unit;
a circular plug arranged at an end of the second electrode;
a handle coupled to the catheter body, an integrated circuit board being arranged inside the handle;
an integrated cable extending from the handle; or
a cable taper coupled to an end of the integrated cable and an RF energy generator.

10. The RF catheter according to claim 1, wherein the catheter body is a hollow, cylindrical catheter provided with at least one marking tape, and a locating ring is sleeved onto and in contact with the catheter body.
